(19) United States
(12) Patent Application Publication
(10) Pub. No.: US 2023/0091022 A1
(43) Pub. Date: Mar. 23, 2023

(54) TOOTH MOVING DEVICE

(71) Applicant: Shuji YAMAGUCHI, Saitama (JP)

(72) Inventor: Shuji YAMAGUCHI, Saitama (JP)

(21) Appl. No.: 17/759,403

(22) PCT Filed: Feb. 3, 2021
(86) PCT No.: PCT/JP2021/003807

§ 371 Date: Jul. 25, 2022

(30) Foreign Application Priority Data
Feb. 6, 2020 (JP) 2020-018495

(51) Int. Cl. A61C 7/10

(52) U.S. Cl. CPC A61C 7/10

(57) ABSTRACT

The present invention provides a tooth moving device, effective for distalizing a posterior tooth or a canine, and also capable of being applied for distalizing not only a maxillary but also mandibular posterior tooth or canine. A tooth moving device 1 of the present invention comprises: a track part 6 extending along a teeth alignment on a maxillary palatal side, or on a maxillary or mandibular buccal side; a sliding part 13 slidably attached to the track part 6; an anchoring part 2 attached at a position close to a distal end relative to the sliding part 13; and an elastic part 14, one end of which is held by the sliding part 13 and the other end of which is held at a position close to the distal end relative to the sliding part 13, and which biases the sliding part 13 toward the distal end 5.

### TECHNICAL FIELD

The present invention relates to a tooth moving device for distalizing (moving toward a back-teeth side) a posterior tooth or a canine (premolar, molar, or canine, especially, premolar or canine).

### BACKGROUND ART

Conventionally, a tooth moving device, such as the one shown in the following Patent Document 1, has been known as a device capable of distalizing a maxillary posterior tooth.

The tooth moving device disclosed in Patent Document 1 comprises: a track part 6 extending from a mesial end 4 to a distal end 5 along a palatal-side teeth alignment; a sliding part 7, slidably attached to the track part 6, and engaging with a holding member 51 configured to be attached to a maxillary posterior tooth; a lock part 8 fixed to the track part 6 close to the mesial end 4 relative to the sliding part 7; an elastic part 9 intervening between the lock part 8 and the sliding part 12 to bias the sliding part 12 toward the distal end; and anchoring parts 2 and 3 which are anchored to an implanted member 53 (see FIG. 2 in the same document) configured to be implanted into a palate and which are connected to the distal end 5, as shown in FIG. 1 in the same document.

With this kind of tooth moving device, the elastic part 9 supported by the lock part 8 applies elastic force to a maxillary posterior tooth, which enables it to be distalized.

### PRIOR ART DOCUMENTS

### Patent Documents

Patent Document 1: JP 6557887 B

### SUMMARY OF THE INVENTION

### Problem to be Solved by the Invention

By the way, the tooth moving device described above requires the lock part 8 to be attached at a position close to the mesial end 4 of the track part 6 relative to a posterior tooth to be distalized, and also requires the elastic part 9 to intervene between the lock part 8 and the sliding part 12. That is, the track part 6 has sufficient length on its mesial side in distalizing a posterior tooth (molar) on a distal side, while the track part 6 does not have enough length on its mesial side in distalizing a posterior tooth (premolar) on a mesial side. Especially in distalizing a first premolar, it can be difficult to attach the lock part 8 and the elastic part 9 due to the shortage of the length on the mesial side of the track part 6. Furthermore, the tooth moving device described above is for distalizing a maxillary posterior tooth, and another means is required for distalizing a mandibular posterior tooth or canine. However, the one that has a sufficient effect as the tooth moving device in Patent Document 1 does has not yet been proposed.

In light of the above, the present invention aims to provide a tooth moving device, which is effective in distalizing a posterior tooth or a canine located on a mesial side, especially such as a first premolar, and which is also capable of being applied in distalizing not only a maxillary but also mandibular posterior tooth or canine.

### Means of Solving the Problem

The present invention is a tooth moving device for distalizing a posterior tooth or a canine, comprising:

a track part, which extends from a mesial end to a distal end along a teeth alignment on a maxillary palatal side, or on a maxillary or mandibular buccal side;

a sliding part, which is slidably attached to the track part, and which is connected to a holding member configured to be attached to the posterior tooth or the canine to be distalized;

an anchoring part, which is connected to the track part at a position close to the distal end relative to the sliding part, and which is attached to a first implanted member configured to be implanted into a jawbone, and/or attached to another posterior tooth located close to the distal end relative to the posterior tooth or the canine; and

an elastic part, one end of which is held by the sliding part and the other end of which is held at a position close to the distal end relative to the sliding part by the track part, the anchoring part, or a second implanted member configured to be implanted into a jawbone, and which biases the sliding part toward the distal end,

wherein the anchoring part is attached to the first implanted member configured to be implanted into the jawbone on a maxillary palatal side, in the case where the track part extends along a teeth alignment on a maxillary palatal side, and

wherein the anchoring part is attached to the first implanted member configured to be implanted into the jawbone on a maxillary or mandibular buccal side and/or is attached to the another posterior tooth, in the case where the track part extends along a teeth alignment on a maxillary or mandibular buccal side.

### Effects of the Invention

In a tooth moving device according to the present invention, a lock part is not required to be attached to a track part at a position close to a mesial end relative to a posterior tooth to be distalized as seen in a conventional tooth moving device. This enables it to be used even in distalizing a posterior tooth or a canine located on a mesial side, such as a first premolar, with no problem. In addition, an anchoring part connected to the track part to fix it to a predetermined position along a teeth alignment may be attached to a first implanted member configured to be implanted into a jawbone or may be attached to another posterior tooth located close to a distal end relative to a posterior tooth or a canine to be distalized, which makes it possible to be applied in distalizing not only a maxillary but also mandibular posterior tooth or canine. Moreover, elastic force can easily be applied to a position near a dental root of a posterior tooth or the like. This enables a posterior tooth or the like to be distalized with its root and all, and brings an effect that a posterior tooth or the like is less likely to get inclined.

### BRIEF DESCRIPTION OF THE DRAWINGS

FIG. 1 is a schematic view of a first embodiment of a tooth moving device according to the present invention.

FIG. 2 is a schematic view of a part of a sliding part, which holds an elastic part.

FIG. 3 is a schematic view of a part of an anchoring part (first anchoring part), which holds an elastic part.

FIG. 4 is a schematic view of a second embodiment of a tooth moving device according to the present invention.

FIG. 5 is a schematic view of a part of an anchoring part (third anchoring part) which holds an elastic part.

### MODE FOR CARRYING OUT THE INVENTION

One example of a tooth moving device according to the present invention is described below with reference to the Drawings. It should be noted that materials, shapes, configurations, manufacturing methods, and the like of each member in the following description are just one example, and they can be appropriately changed to the ones that bring the same effects as those from the present invention. Thus, the present invention also includes the case where something made of metal in the following description is changed to the one made of another material (such as synthetic resin), for example.

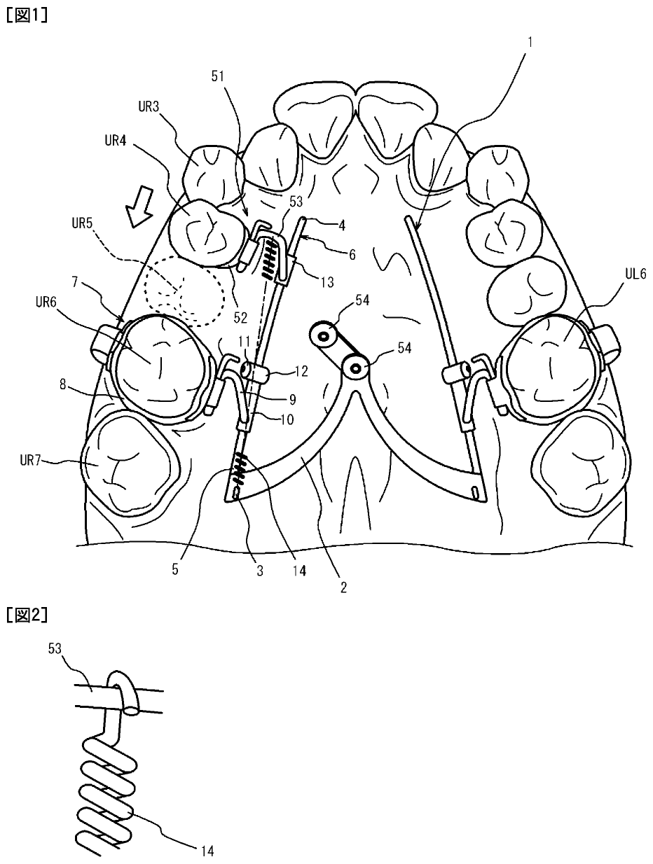

FIG. 1 relates to a first embodiment of the tooth moving device and is a view showing it being attached to a patient's upper jaw from a viewpoint facing toward a palate. The teeth to which reference characters are given in FIG. 1 are a maxillary right canine UR3, a maxillary right first premolar UR4, a maxillary right second premolar UR5, a maxillary right first molar UR6, a maxillary right second molar UR7, and a maxillary left first molar UL6. FIG. 1 also shows the state where the maxillary right first premolar UR4 is being distalized and the maxillary right second premolar UR5 is removed.

For describing a tooth moving device 1 according to the present embodiment, a holding member 51 and an anchor screw (corresponding to “a first implanted member” in the present specification) are described first.

The holding member 51 is configured to be attached to a posterior tooth or a canine to be distalized. The holding member 51 of the present embodiment is formed by integrally connecting a metallic plate part 52 and a metallic wire part 53. For connecting the plate part 52 and the wire part 53, they may be welded together or may be connected by swaging, for example. Meanwhile, the plate part 52 and the wire part 53 may be integrally formed from the beginning along with a sliding part 13 described later by using a 3D printer or the like. A molding method of a 3D printer is not particularly limited including the following description below, and it may be selective laser sintering (laser sintering), stereolithography apparatus, or inkjet, for example. In addition, the wire part 53 may be hooked on a tubular part provided on the plate part 52. The holding member 51 described above is attached by fixing the plate part 52 to a palatal side of the maxillary right first premolar UR4 with adhesive agent. Moreover, a configuration of the holding member 51 is not limited to this, and for example, a band-shaped member surrounding a posterior tooth or the like to be distalized may be used instead of the plate part 52.

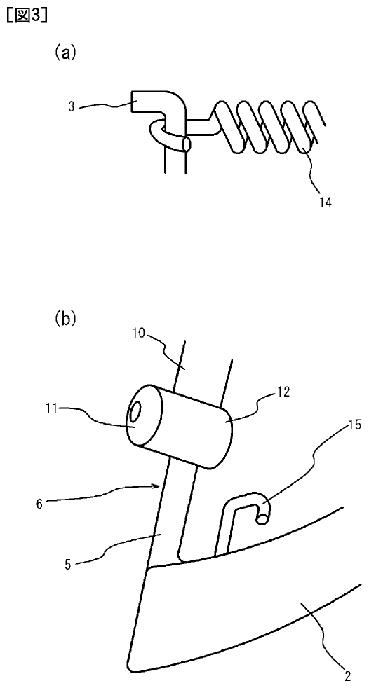

The anchor screw is a member configured to be implanted into a jawbone. Although the anchor screw in FIG. 1 is unshown because it is implanted into a premedial part of a palate, it is formed into a generally external-thread shape as with the member referred to as 53 in FIG. 2 in Patent Document 1. In addition, a head of the anchor screw has an internal-thread part, and a low-head fixation screw 54 shown in FIG. 1 is attached to the internal-thread part. Although a total of two anchor screws are used in the present embodiment, the number is not limited to this but also may be one, or three or more.

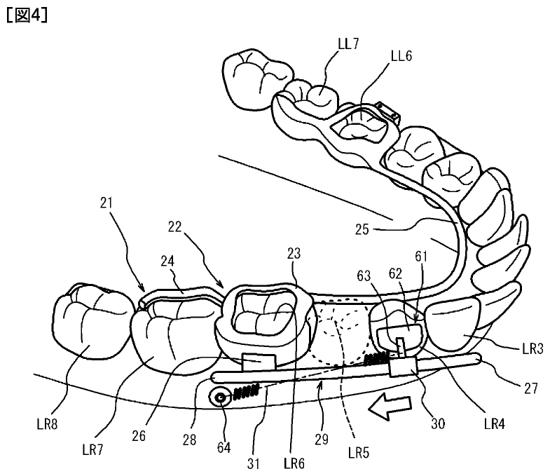

Next, a tooth moving device 1 according to the present embodiment is described. The tooth moving device 1 according to the present embodiment has an anchoring part (first anchoring part) 2 which is coupled with the anchor screw by tightening the fixation screw 54. The first anchoring part 2 of the present embodiment is formed of a metallic plate. It has two unshown holes at positions corresponding to the two implanted anchor screws on its mesial side and is forked on its distal side. The first anchoring part 2 can be fixed to a palate by inserting the fixation screws 54 into the two holes on the mesial side and tightening them on the anchor screws. Also, an L-shaped hook part 3 shown in FIG. 3(a) is provided on the distal side of the first anchoring part 2.

The tooth moving device 1 further has a rod-shaped track part 6 extending from a mesial end 4 to a distal end 5 along a palatal teeth alignment. The track part 6 of the present embodiment is provided on each of left and right sides. Each distal end 5 connects to a left or right end of the forked first anchoring part 2. The track part 6 of the present embodiment is formed of a metallic wire having a circular cross-section, and is integrated with the first anchoring part 2 by welding them together. In the present embodiment, a wire with a diameter of approximately 0.7 to 1.5 mm is used. A wire used as the track part 6 can be adopted regardless of its kind and may have a polygonal or elliptical cross-section, for example. Furthermore, not only solid one but also hollow one may be adopted. In addition, the first anchoring part 2 and the track part 6 may be integrally formed from the beginning with a 3D printer, for example, and a method for forming them can be changed appropriately.

A different anchoring part (second anchoring part) 7 from the first anchoring part 2 is provided near the distal end 5 of the track part 6. The second anchoring part 7 of the present embodiment has a metallic band part 8 which is configured to be attached to the maxillary right first molar UR6 so as to surround it, and a connecting part 9 which is formed of a metallic wire and connected on a palatal side of the band part 8. The connecting part 9 is also connected to a tubular part 10 which is in a tubular shape and into which the track part 6 is inserted. On a mesial side of the tubular part 10, a fixed part 12 to be fixed to the track part 6 by tightening a screw 11 is provided. Removing the maxillary right second premolar UR5 allows the maxillary right first molar UR6 to move toward the mesial side easily, which could interfere with the distalization of the maxillary right first premolar UR4. However, the band part 8 attached to the maxillary right first molar UR6 is connected to the tubular part 10 via the connecting part 9, and the fixed part 12 prevents the tubular part 10 from moving toward the mesial side. That can prevent the maxillary right first molar UR6 from moving toward the mesial side. Instead of the band part 8, a plate-shaped thing configured to be fixed to the maxillary right first molar UR6 with adhesive agent may be used, for example. Also, the band part 8, the connecting part 9, and the tubular part 10 may be integrally formed from the beginning with a 3D printer, for example. Although the tubular part 10 and the fixed part 12 of the present embodiment are separate members from each other, they may be integrated together (for example, by welding them which are separately formed, or by integrating them from the beginning with a 3D printer). Integrating the tubular part 10 with the fixed part 12 as described above allows the track part 6 to be also held by the second anchoring part 7 in addition to the first anchoring part 2, which enables the track part 6 to be more securely held at a desired position. Also, since the second anchoring part 7 is supported by the rigid first anchoring part 2 formed of a plate, it can be used for correcting the maxillary right first molar UR6 tilting toward a buccal side or a palatal side.

Additionally, a tubular sliding part 13 is slidably attached near the mesial end 4 of the track part 6. The sliding part 13 of the present embodiment is welded to an end of the wire part 53 described above and integrated with the holding member 51.

The tooth moving device 1 further has a coiled spring (elastic part) 14 which biases the sliding part 13 toward the distal end 5. Both ends of the coiled spring 14 of the present embodiment are curved in an arc-shape. One end of the coiled spring 14 is hooked on the hook part 3 as shown in FIG. 3(a). The other end of the coiled spring 14 is hooked on the wire part 53 as shown in FIG. 2 while the coiled spring 14 is stretched. Not being shown in figures, a hook part (such as a hook part 15 shown in FIG. 3(b) described below) may be provided on the sliding part 13 and the other end of the coiled spring 14 may be hooked on this hook part. It should be noted that the shapes of the both ends of the coiled spring 14 are not limited to the arc-shape as shown but also may be in a ring-shape, for example.

With the tooth moving device 1 according to the present embodiment having the configuration described above, a force of the coiled spring 14 also acts on the maxillary right first premolar UR4 to which the holding member 51 is attached when the sliding part 13 is being biased toward the distal end 5 by the coiled spring 14 while being guided by the track part 6. This enables distalization of the maxillary right first premolar UR4 along the direction in which the track part 6 extends. In addition, as only the sliding part 13 is provided near the mesial end 4 of the track part 6 as shown in FIG. 1, the track part 6 has a length to spare near the mesial end 4. Thus, for example, even in the case where the sliding part 13 is required to be moved toward the side of the mesial end 4 in treatment, it can be dealt without remaking the track part 6. Moreover, due to the spare length near the mesial end 4 of the track part 6, even the maxillary right canine UR3 located on the mesial side relative to the maxillary right first premolar UR4 can be distalized with no problem.

A configuration for attaching the coiled spring 14 can be changed appropriately. For example, the J-shaped hook part 15 shown in FIG. 3(b) may be provided instead of the hook part 3 shown in FIG. 1 and FIG. 3(a), and an end of the coiled spring 14 may be hooked on the hook part 15. Although the shown hook part 15 has a form projecting from the first anchoring part 2 toward the mesial side, it may have a form projecting toward the distal side or laterally projecting toward a teeth alignment. Moreover, as shown in FIG. 3(b), the fixed part 12 may be added on the distal side of the tubular part 10, and an end of the coiled spring 14 may be held between the screw 11 and the fixed part 12 in fixing the fixed part 12 to the track part 6 by tightening the screw 11. Furthermore, an end of the coiled spring 14 may be fixed by winding another wire rod around it (or may be fixed by swaging), and this wire rod may be wound around the fixed part 12 to fix it. In this case, a groove may be provided around the fixed part 12 so that the wire rod can be wound along it. Also, an anchor screw (corresponding to “a second implanted member” in the present specification) may be implanted into a palate near the distal end 5 of the track part 6 so that an end of the coiled spring 14 may be held using this anchor screw, for example.

Although the forked first anchoring part 2 is used in the tooth moving device 1 shown in FIG. 1, a first anchoring part 2 having only one side (side on which a posterior tooth or a canine to be distalized is located) may be used. Although the second anchoring part 7 of the present embodiment is attached to the maxillary right first molar UR6 and the maxillary left first molar UL6, one side of them may be attached to another posterior tooth (for example, attached to the maxillary right second molar UR7 and the maxillary left first molar UL6), or may only be attached to either of them. Preferably, the first anchoring part 2 and the second anchoring part 7 should be used in combination as in the present embodiment in order to stably hold the track part 6, but only either of them may be used.

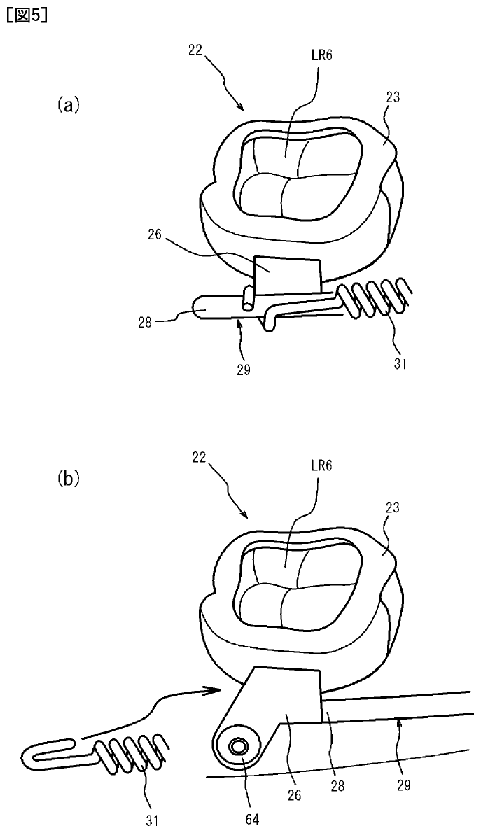

Next, a second embodiment of a tooth moving device according to the present invention is described with reference to FIG. 4. FIG. 4 shows a tooth moving device 21 attached to a patient's lower jaw. The teeth to which reference characters are given in FIG. 4 are a mandibular right canine LR3, a mandibular right first premolar LR4, a mandibular right second premolar LR5, a mandibular right first molar LR6, a mandibular right second molar LR7, a mandibular right third molar LR8, a mandibular left first molar LL6, and a mandibular left second molar LL7. FIG. 4 also shows the state where the mandibular right first premolar LR4 is being distalized and the mandibular right second premolar LR5 is removed.

As shown in FIG. 4, a holding member 61 is configured to be attached to the mandibular right first premolar LR4 to be distalized. The holding member 61 of the present embodiment is formed by integrally connecting a metallic plate part 62 and a metallic wire part 63. The wire part 63 is integrally connected to a sliding part 30 described below, and at the time of manufacture, the plate part 62, the wire part 63, and the sliding part 30 are integrally formed. The holding member 61 described above is attached by fixing the plate part 62 to a buccal side of the mandibular right first premolar LR4 with adhesive agent.

In addition, an unshown anchor screw (corresponding to “a second implanted member” in the present specification), which is configured to be implanted into a buccal side of a mandibular jawbone and to which a low-head fixation screw 64 can be attached, is implanted between and below the mandibular right first molar LR6 and the mandibular right second molar LR7 as shown in FIG. 4. The anchor screw may be provided at a position other than the shown position (for example, may be provided between the mandibular right second molar LR7 and the mandibular right third molar LR8).

The tooth moving device 21 has an anchoring part (third anchoring part) 22 configured to be attached to the mandibular right first molar LR6 and the mandibular right second molar LR7 and to the mandibular left first molar LL6 and the mandibular left second molar LL7 in the present embodiment. The third anchoring part 22 is formed of a metallic plate and has a ring part 23 which is configured to be attached to the mandibular right first molar LR6 (mandibular left first molar LL6) so as to surround it, and an extending part 24 which is integrally connected to the ring part 23 as well as extends on a lingual side of the mandibular right second molar LR7 (mandibular left second molar LL7). Furthermore, the two ring parts 23 are integrated together on their mesial ends with an approximately U-shaped connecting wire part 25 which is formed of a metallic wire as well as extends along a lingual teeth alignment. A connecting plate part 26 which is integrally connected to a track part 29 described below is provided on a buccal side of the ring part 23 which is configured to be attached to the mandibular right first molar LR6.

The tooth moving device 21 further has a rod-shaped track part 29 extending from a mesial end 27 to a distal end 28 along a buccal teeth alignment. The track part 29 of the present embodiment is formed of a metallic wire having a circular cross-section. In addition, the track part 29 is connected to the connecting plate part 26 by welding, for example, and held by the third anchoring part 22. The track part 29 may be directly connected to the third anchoring part 22 without the connecting plate part 26. A wire with a diameter of approximately 0.7 to 1.5 mm is used as the track part 29 of the present embodiment. A wire used as the track part 29 can be adopted regardless of its kind and may have a polygonal or elliptical cross-section, for example. Furthermore, not only solid one but also hollow one may be adopted. In addition, the third anchoring part 22 (ring part 23, extending part 24), the connecting wire part 25, the connecting plate part 26, and the track part 29 may be integrally formed from the beginning (which may be all integrated or several of which may be partly integrated) with a 3D printer, for example, and a method for forming them can be changed appropriately.

Additionally, a tubular sliding part 30 is slidably attached near the mesial end 27 of the track part 29. The sliding part 30 of the present embodiment is welded to an end of the wire part 63 described above and integrated with the holding member 61.

The tooth moving device 21 further has a coiled spring (elastic part) 31 which biases the sliding part 30 toward the distal end 28. As with the coiled spring 14 shown in FIG. 2 and FIG. 3(a), both ends of the coiled spring 31 of the present embodiment are also curved in an arc-shape. One end of the coiled spring 31 is attached to an anchor screw (unshown), which is implanted between the mandibular right first molar LR6 and the mandibular right second molar LR7, with the fixation screw 64. The other end of the coiled spring 31 is hooked on the wire part 63 while the coiled spring 31 is stretched.

With the tooth moving device 21 according to the present embodiment, a biasing force of the coiled spring 31 acts on the mandibular right first premolar LR4 via the sliding part 30 and the holding member 61, which enables the mandibular right first premolar LR4 to be distalized along the direction in which the track part 29 extends. As only the sliding part 30 is provided near the mesial end 27 of the track part 29 also in the tooth moving device 21 according to the present embodiment, the track part 29 has a length to spare near the mesial end 27. Thus, even in the case where the sliding part 30 is required to be moved toward the side of the mesial end 27, it can be dealt without remaking the track part 29. Moreover, due to the spare length near the mesial end 27 of the track part 29, the mandibular right canine LR3 can be distalized with no problem.

The tooth moving device 21 according to the present embodiment can also be changed appropriately in such a way as to fulfill its functions. For example, for attaching the coiled spring 31, it may be hooked on the distal end 28 of the track part 29 instead of the above-mentioned anchor screw as shown in FIG. 5(a). The third anchoring part 22 can also be changed appropriately as shown in FIG. 5(b). For example, a shape of the connecting plate part 26 may be changed, an anchor screw may be implanted into a buccal side of a mandibular jawbone at a position where the connecting plate part 26 is located, and the connecting plate part 26 may be fixed to the anchor screw with the fixation screw 64. In this case, an end of the coiled spring 31 may be made into a long-folded shape, and this end may be hooked on the connecting plate part 26 to hold the coiled spring 31. Furthermore, a hook part (such as the hook part 15 shown in FIG. 3(b)) may be provided on the connecting plate part 26 or the track part 29, and this hook part may be used to hold the coiled spring 31. For holding the track part 29 at a predetermined position, only the above-mentioned anchor screw implanted into the buccal side of the mandibular jawbone may be used without using the third anchoring part 22.

The track part 29 of the tooth moving device 21 shown in FIG. 4 extends along a buccal teeth alignment. However, the track part 29 can be configured to extend along a lingual teeth alignment by using the connecting wire part 25 as the track part 29, for example. Moreover, something having a shape similar to the track part 29 shown in FIG. 4 may be extended along a lingual teeth alignment. In the case where the track part 29 is extended along a lingual teeth alignment, the third anchoring part 22 attached to the mandibular left first molar LL6 or the like may be used to hold it, or an anchor screw implanted into a lingual side of a mandibular jawbone may be used. Furthermore, the tooth moving device 21 can also be used for distalizing a maxillary posterior tooth or canine by configuring the track part 29 to extend along a maxillary teeth alignment (which may be either on a palatal side or on a buccal side).

One embodiment of the present invention is described above. However, the present invention is not limited to the specific embodiment, but also includes various modifications within the intent of the present invention described in the scope of the claims unless otherwise limited in the above description. Also, the effects in the above-mentioned embodiments are merely examples of the effects derived from the present invention and does not mean that the effects of the present invention are limited to the above-mentioned effects.

The present specification discloses at least the following inventions.

[1] A tooth moving device for distalizing a posterior tooth or a canine, comprising:

a track part, which extends from a mesial end to a distal end along a teeth alignment;

a sliding part, which is slidably attached to the track part, and which is connected to a holding member configured to be attached to the posterior tooth or the canine to be distalized;

an anchoring part, which is connected to the track part at a position close to the distal end relative to the sliding part, and which is attached to a first implanted member configured to be implanted into a jawbone, and/or attached to another posterior tooth located close to the distal end relative to the posterior tooth or the canine; and an elastic part, one end of which is held by the sliding part and the other end of which is held at a position close to the distal end relative to the sliding part by the track part, the anchoring part, or a second implanted member configured to be implanted into a jawbone, and which biases the sliding part toward the distal end.

[2] The tooth moving device according to [1],

wherein the track part extends along a teeth alignment on a maxillary palatal side; and

wherein the anchoring part is attached to the first implanted member configured to be implanted into the jawbone on a maxillary palatal side.

[3] The tooth moving device according to [1],

wherein the track part extends along a teeth alignment on a maxillary or mandibular buccal side; and

wherein the anchoring part is attached to the first implanted member configured to be implanted into the jawbone on a maxillary or mandibular buccal side and/or is attached to the another posterior tooth.

[4] The tooth moving device according to [1],

wherein the track part extends along a teeth alignment on a mandibular lingual side; and

wherein the anchoring part is configured to be attached to the another posterior tooth.

### DENOTATION OF REFERENCE NUMERALS

1 tooth moving device
2 first anchoring part (anchoring part)
4 mesial end
5 distal end
6 track part
7 second anchoring part (anchoring part)
13 sliding part
14 coiled spring (elastic part)
21 tooth moving device
22 third anchoring part (anchoring part)
27 mesial end
28 distal end
29 track part
30 sliding part
31 coiled spring (elastic part)
51 holding member
61 holding member

What is claimed is:

1. A tooth moving device for distalizing a posterior tooth or a canine, comprising:
a track part, which extends from a mesial end to a distal end along a teeth alignment on a maxillary palatal side, or on a maxillary or mandibular buccal side;
a sliding part, which is slidably attached to the track part, and which is connected to a holding member configured to be attached to the posterior tooth or the canine to be distalized;
an anchoring part, which is connected to the track part at a position close to the distal end relative to the sliding part, and which is attached to a first implanted member configured to be implanted into a jawbone, and/or attached to another posterior tooth located close to the distal end relative to the posterior tooth or the canine; and
an elastic part, one end of which is held by the sliding part and the other end of which is held at a position close to the distal end relative to the sliding part by the track part, the anchoring part, or a second implanted member configured to be implanted into a jawbone, and which biases the sliding part toward the distal end,
wherein the anchoring part is attached to the first implanted member configured to be implanted into the jawbone on a maxillary palatal side, in the case where the track part extends along a teeth alignment on a maxillary palatal side, and
wherein the anchoring part is attached to the first implanted member configured to be implanted into the jawbone on a maxillary or mandibular buccal side and/or is attached to the another posterior tooth, in the case where the track part extends along a teeth alignment on a maxillary or mandibular buccal side.
